(19) United States
(12) Patent Application Publication
(10) Pub. No.: US 2016/0081309 A1
(43) Pub. Date: Mar. 24, 2016

(54) FRUCTOBACILLUS AS A PROBIOTIC FOR HONEYBEES

(71) Applicant: Indiana University Research & Technology Corporation, Indianapolis, IN (US)

(72) Inventor: Irene Newton, Bloomington, IN (US)

(73) Assignee: Indiana University Research & Technology Corporation, Indianapolis, IN (US)

(21) Appl. No.: 14/860,463

(22) Filed: Sep. 21, 2015

Related U.S. Application Data
(60) Provisional application No. 62/052,976, filed on Sep. 19, 2014.

(51) Int. Cl. A01K 47/02; C12R 1/01; C12N 1/20

(52) U.S. Cl. CPC A01K 47/02; C12N 1/20; C12R 1/01

(57) ABSTRACT

Described herein are methods for promoting microbiome development in honey bees using an effective amount of Fructobacillus as a probiotic. Also described are compositions comprising Fructobacillus strains capable of promoting microbiome development in honey bees, and processes for making such compositions.

### CROSS-REFERENCE TO RELATED APPLICATIONS

This application claims priority to U.S. Provisional Application No. 62/052,976, filed on Sep. 19, 2014, the entire disclosure of which is expressly incorporated herein by reference.

### STATEMENT REGARDING FEDERALLY SPONSORED RESEARCH

This invention was not made with government support.

### BACKGROUND OF THE INVENTION

The European honey bee (Apis melliferia) is heavily utilized in agriculture for both pollination efforts and for the production of honey. The decline in population size of this agricultural insect has increased both interest and research into microbial communities naturally populating the honey bee.

The gut of the European honey bee is host to a characteristic microbial community composed predominantly of three major phyla (Firmicutes, Proteobacteria, and Actinobacteria) within which several honey bee specific families and genera are taxonomically classified. The core microbiome of the adult bee gut has been characterized as being composed of a small number of bacterial clades, some with genus and species designations. These core clades (and named genera), found within the previously mentioned bacterial phyla, are as follows: Firm-4, Firm-5 (within the Firmicutes), Bifido (within the Actinobacteria), and Alpha-2.1, Alpha-2.2 (Parasaccharibacter sp.), Alpha-1, Beta (Snodgrassella), Gamma-1 (Gilliamella sp.) and Gamma2 (Frischella sp.)(within the Proteobacteria).

The development of a honey bee microbiome inclusive of these core clades requires interaction between kin and/or with hive components. The honey bee is a eusocial insect that lives in a dense population of individuals that make up the colony. The worker caste of bees performs different tasks in the hive, dependent on their age. Younger bees (“nurse bees”) are generally constrained to the hive, feed on protein and lipid rich processed pollen (“bee bread”) and participate in the rearing of brood. Older bees (“foragers”) fly out of the hive in search of nectar and pollen. When food is brought back to the hive, it is passed from bee to bee via trophallaxis, a mechanism for food exchange, and made into the food products honey and bee bread. Lactobacillus sp. commonly associated with pollen have been identified in the crop of adult honey bees. Full transmission of the characteristic gut microbiota requires the physical interaction of honey bees with hive environments and with fecal material, and cannot be completed through trophallaxis alone. Natural hive rearing, including interactions with other bees and hive components, is important to the colonization by gram-negative core bacteria (such as Gilliamella species) while exposure to comb or trophallaxis alone resulted in gut communities that contained other core microbial members (Firm-4, Firm-5, Gamma-2, Bifido, Snodgrassella, Alpha-2.1).

Directional change in species composition in an environment, over time, is referred to as ecological succession. Young bees are relatively uncolonized, with the first members to colonize the gut often being facultative anaerobes, such as Escherichia coli. These “pioneering species” pave the way for colonization of the gut by obligate anaerobes by consuming oxygen, producing carbon dioxide, and changing the pH. During early stages of succession, bacterial diversity is often low and the community changes rapidly; this result has been observed in the mammalian digestive tract and also in the insect gut and in other, non-host associated environments. However, after this period of early succession, the bacterial community reaches a steady state often referred to as the “climax community.” The climax community of a bacterial population would be reached when there is an equilibrium that can be maintained of a specific mix of bacteria. The event often coincides with a later developmental stage of the organism harboring the bacterial community. Bacterial diversity and the total number of bacteria are higher in a climax community than during early succession.

The honey bee is a holometabolous insect and moves through a life cycle marked by the metamorphic transition from larval stage into a developed imago form. During the course of this event, a matured larva is enclosed in its brood cell by worker bees, pupates, and develops into a honey bee. During this metamorphic period, the larval gut is shed. Consequently, newly enclosed worker bees (NEWs) retain none of the characteristic microbiota associated with the larval gut and, over the span of a few days, are colonized with bacterial phylotypes characteristic of an adult honey bee. However, because larval bees mature and pupate in the same space—the brood cell—it is possible that they are re-inoculated with the same microbes they were exposed to as larvae upon completion of their metamorphic transition. Additionally, because NEWs interact with hive components such as comb and processed food, colonization of these hive components by bacterial community members may impact community succession.

### SUMMARY OF THE INVENTION

Described herein are methods for promoting microbiome development in honey bees using an effective amount of Fructobacillus as a probiotic. Also described are compositions comprising Fructobacillus strains capable of promoting microbiome development in honey bees, and processes for making such compositions.

In a particular aspect provided herein is a method of promoting microbiome development in honey bees, comprising providing an effective amount of one or more strains of Fructobacillus capable of promoting microbiome development in honey bees, or a supernatant thereof, to a honey bee colony. In certain aspects, at least one of the one or more strains of Fructobacillus is resistant to tetracycline. The one or more strains can be exogenous to the honey bee colony, or endogenous to the colony.

The one or more strains of Fructobacillus is provided to the honey bee colony by at least one technique chosen from feeding the one or more strains of Fructobacillus to the honey bees of the honey bee colony, and applying the one or more strains of Fructobacillus to the frames of a hive. The one or more strains of Fructobacillus can be formulated into a composition prior to being fed or applied.

In another particular aspect provided herein is a composition comprising one or more strains of Fructobacillus capable of promoting microbiome development in honey bees, or a supernatant thereof, and a carrier. The carrier can be a liquid carrier or a gel-based carrier.

In certain aspects, the composition further comprises at least one carbon source chosen from sucrose, fructose, and glucose.

In another particular aspect provided herein is a process for manufacturing the composition described herein. The process generally comprises culturing one or more strains of Fructobacillus capable of promoting microbiome development in honey bees, and combining at least one of the obtained cultures or a supernatant thereof, with a carrier chosen from a liquid carrier and a gel-based carrier, and at least one carbon source chosen from sucrose, fructose, and glucose into a homogenous composition.

In certain aspects, one of the one or more strains of Fructobacillus is Fructobacillus strain FJL, a deposit of which is maintained by Dr. Irene Newton, Department of Biology, Indiana University, Bloomington, 107 S. Indiana Ave, Bloomington, Ind. 47405. In other aspects, one of the one or more strains of Fructobacillus is a tetracycline resistant mutant of Fructobacillus strain FJL.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIG. 1: Schematic depicting method used to isolate Fructobacillus sp. and identify its prevalence in the hive environment and on the honey bee during development.

FIG. 2: Diagram depicting phylogenetic analysis of top ten bacterial species, based upon 16s rRNA gene sequences. Maximum likelihood tree constructed using a Jukes-Cantor correction model, with 1000 bootstrap replicates. Sequence names beginning with “AB” or “HM” are published sequences taken from a honey bee specific training set (Newton and Roeselers, (2012) Bmc Microbiol, 12).

FIGS. 3A-3B: Graphs depicting sequence abundance of top ten lactic acid bacteria (LAB) species, cultured from each subsampled honey bee environment, in three hives, binned at 99% identity based upon 16s rRNA sequencing. FIG. 3A) Average species (OTU) abundances for each sampled environment for each of the top LABs. Data compiled and averaged from each of three sampled hives. Error bars are the result of three independent biological replicates. FIG. 3B) Sequence abundance for top ten OTUs found in each of three sampled hives across four environments.

FIG. 4: Schematic showing that the Cell acts as a centralized hub, connecting OTUs between bee-associated environments. Network generated through pairwise examination of environments sharing identical species. Edges weighted based on proportion of total sequence abundance observed in that interaction within the subsampled data set. Metrics are not weighted. Connectivity measurements (the number of shared bacterial species between each environment) show that the honey bee-associated environments sampled are all equally well connected. Centrality measurements (the number of shortest paths from each other environment to all others that pass through this particular node) show that the cell serves as a hub, through which bacteria may be transferred across environments.

FIG. 5: Co-culture of Fructobacillus sp. with honey bee-associated microbes. When honey bee-associated microbes are grown with Fructobacillus or with spent medium from Fructobacillus, they exhibit a growth advantage compared to growth alone. FIG. 5A) Phylogeny and heat map of isolates used in a co-culture interaction assay. Change in optical density from expected values plotted as a heat map (yellow=more growth than expected; blue=less growth than expected). Lactobacillus and Fructobacillus isolates significantly increased growth of a Firm-5 isolate. FIG. 5B) Optical density measurements suggest Firm-5 grows more robustly in Fructobacillus spend medium (SM) than in de Man, Rogosa and Sharpe medium (MRS) (t=11.196, df=4, p<0.001). FIG. 5C) The difference in optical density corresponds to a dramatic difference in colony numbers when Firm-5 is grown on MRS plates post incubation for 48 hours in MRS or SM (10⁶ dilutions of cultures plated in triplicate).

### DETAILED DESCRIPTION

Although there has been substantial investment into profiling the microbes most commonly associated with the honey bee gut, research directed towards determining the microbes commonly associated with honey bee-related environments has been more limited in scope. As described herein, the environments from which honey bees can be inoculated and interactions between bacterial community members that shape the microbiome were investigated. Although workers shed their gut lining during metamorphosis, bacteria present during the developmental process, in the food produced by the bees, or in the comb, can persist or facilitate colonization by core microbiome members, such as isolates from core clades Firm-4, Firm-5 (within the Firmicutes), Bifido (within the Actinobacteria), and Alpha-2.1, Alpha-2.2 (Parasaccharibacter sp.), Alpha-1, Beta (Snodgrassella), Gamma-1 (Gilliamella sp.) and Gamma2 (Frischella sp.)(within the Proteobacteria). A subset of the bacterial community was cultured and sequenced. Specifically, the lactic acid bacteria (LAB), an abundant and ubiquitous clade of microbes found associated with bees throughout development and in other external hive environments, were cultured and sequenced. The LAB community composition was examined in environments from across the hive and in the honey bees themselves. Examination of pairwise comparisons between environments containing identical 99% OTUs (bacterial species) showed a homogenous distribution of microbes between the hive environments.

A strain of Fructobacillus capable of promoting the growth of honey bee-specific LABs was identified in the microbial hubs of the brood cell and bee bread. The identified strain, Fructobacillus FJL, was identified as a pioneering species in the development of the honey bee gut community. Described herein are methods for promoting microbiome development in honey bees using an effective amount of Fructobacillus as a probiotic. Also described are compositions comprising Fructobacillus strains capable of promoting microbiome development in honey bees, and processes for making such compositions.

### GENERAL DESCRIPTION

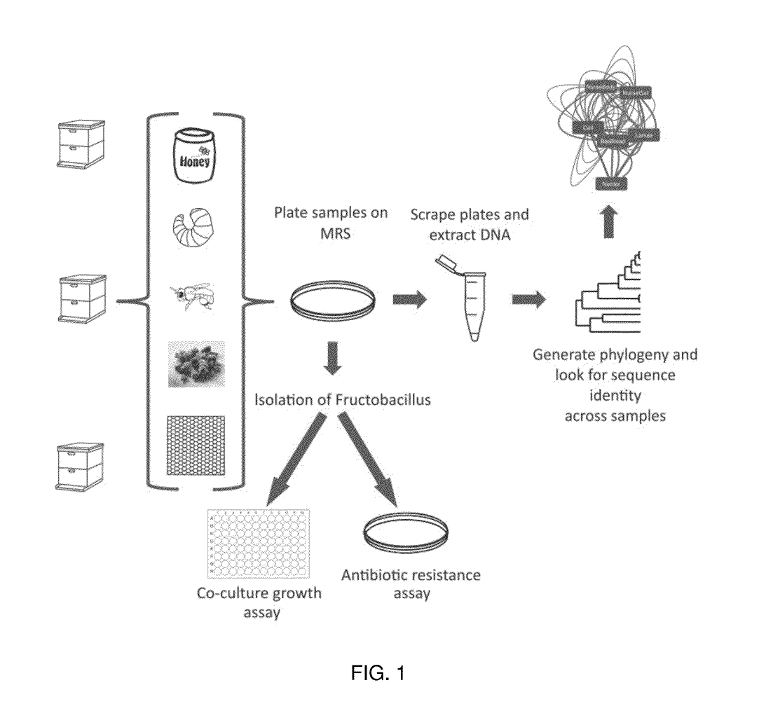

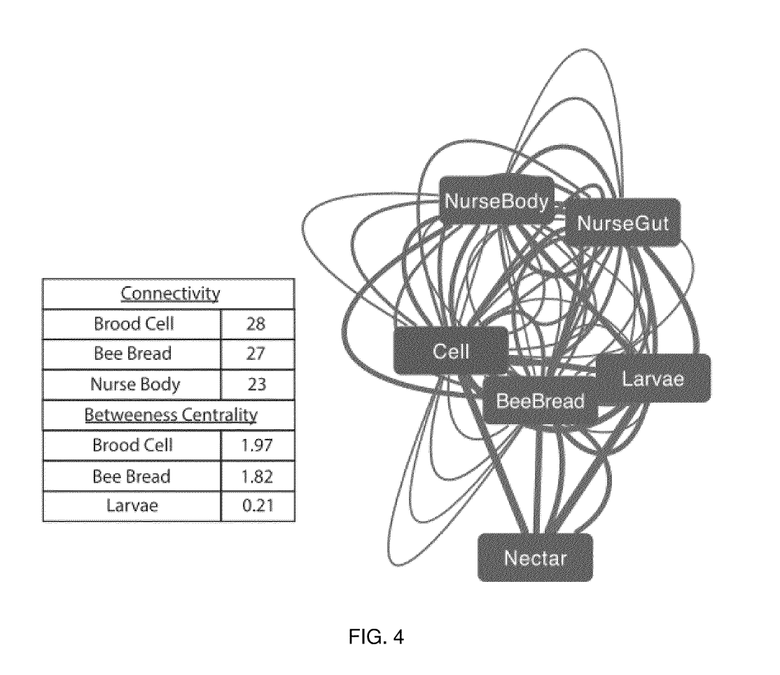

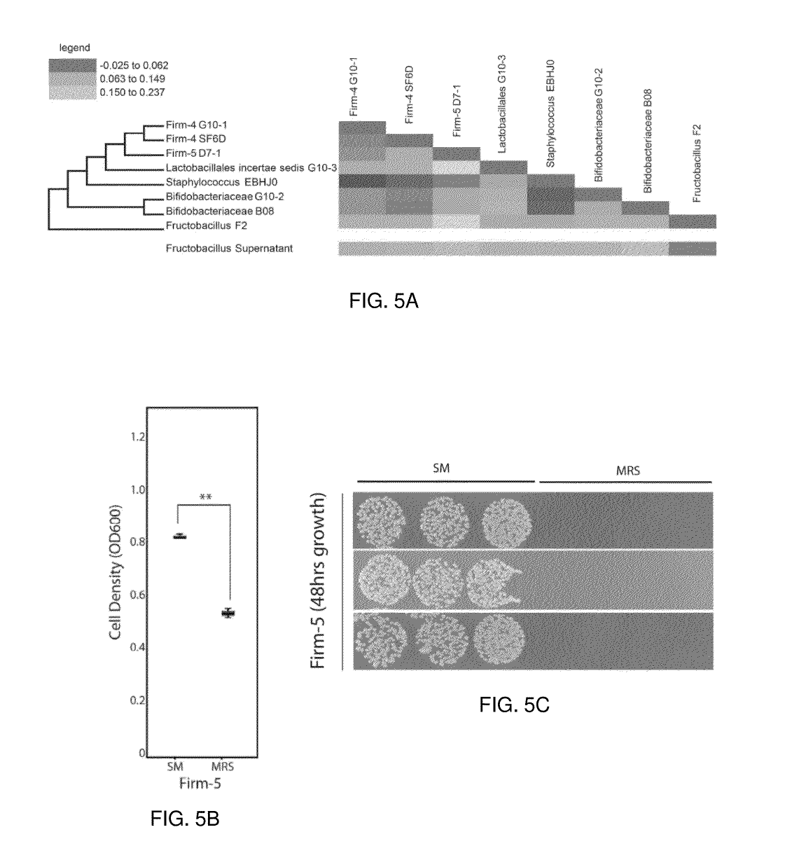

In contrast to the honey bee gut, which is comprised of a few characteristic bacterial clades, the hive of the honey bee is home to a diverse array of microbes, including many lactic acid bacteria (LAB). LAB communities found across hive environments were sampled and analyzed using culture techniques combined with sequencing (FIG. 1). Network analysis was employed to identify microbial hubs sharing nearly identical Operational Taxonomic Units (OTUs), indicating co-occurrence of bacteria between environments (FIG. 4). OTU's are groups of bacterial species that are determined to be similar or related based on shared sequence identity as determined by 16s rRNA sequencing. Through this analysis, which is described in detail throughout the Examples, Fructobacillus was found to colonize both brood cells and bee bread as a pioneering species, establishing an environment conducive to the inoculation by honey bee core bacteria. Co-culture assays showed that the non-core Fructobacillus strain FJL promotes the growth of honey bee specific bacterial species (FIG. 5). Fructobacillus FJL byproducts in spent medium significantly enhanced the growth of the Firm-5 honey bee specific clade (FIG. 5).

Core microbiome development in honey bees can be promoted by providing and effective amount of Fructobacillus as a probiotic to a honey bee colony. An effective amount of the microbial strain (i.e., a probiotic) described herein is an amount that achieves a desired result (e.g., improved growth of core microbiome) in honey bees of a colony. An effective amount can be provided in a single feeding or application, or over time. An effective amount can depend on several factors, such as colony size, method of feeding, and desired effect. An effective amount necessary to achieve a desired result can be determined or modified by one of skill in the art.

Fructobacillus useful as a probiotic are those strains capable of promoting microbiome development in honey bees. Particular strains useful as a probiotic can be identified through methods such as those described in the Examples. For example, Fructobacillus isolates can be co-cultured along with isolates of honey bee core microbiome clades such as, for example, Firm-4 and Firm-5. Fructobacillus strains useful as a probiotic can be identified where growth of the core microbiome isolate is greater than that expected when cultured on its own. Methods of measuring bacterial growth are well known within the art, and include, for example, measuring growth as a function of optical density (methods described in Example I).

A single Fructobacillus strain can be provided to a honey bee colony as a probiotic, or a combination of strains can be provided. Honey bees are commonly prophylactically treated with oxytetracycline for the prevention of foulbrood diseases caused by the bacteria Melissococcus plutonius and Paenibacillus larvae. Therefore, where oxytetracycline is to be applied to a honey bee hive, at least one of the probiotic strains of Fructobacillus provided to the colony is resistant to tetracycline. Tetracycline resistant strains have the benefit of being able to support microbiome development in the honey bee despite application of antibiotic, whereas non-resistant strains that are either provided in methods described herein, or that occur naturally within the hive's environments, would be eliminated and unable to provide any probiotic effect to the developing core microbiome.

Fructobacillus strains capable of promoting core microbiome development in honey bees can be derived from a particular colony's hive (endogenous). An endogenous probiotic strain can be multiplied through culture and returned to the hive to promote microbiome development. Alternatively, an endogenous probiotic strain can be identified and isolated, and then subjected to selection for antibiotic resistance. Particularly, the isolated strain undergoes selection for tetracycline resistance. Selection can be performed, for example, by culturing an isolated colony of the probiotic strain on media containing tetracycline. Colonies found to grow successfully on the tetracycline-containing media can then be selected and used to grow up sufficient numbers to provide to a honey bee colony.

Fructobacillus strains capable of promoting core microbiome development in honey bees can also be derived from another colony's hive, or another source altogether (exogenous). Exogenous strains found capable of promoting core microbiome development can be grown up to sufficient numbers to be provided to a honey bee colony. Where the exogenous probiotic strain(s) is derived from another colony's hive, the hive can be from the same apiary as the hive which is to be provided the probiotic strain(s), or from another apiary. Where the exogenous probiotic strain(s) is derived from another source altogether, Fructobacillus can be isolated from, for example, flowers and fruits. Strains of Fructobacillus useful as a probiotic for honey bees may be found on those flowers from which the bees collect pollen. Once an exogenous strain of Fructobacillus has been identified as capable of promoting core microbiome development in honey bees, it can be subjected to selection for antibiotic resistance, including tetracycline resistance, as described above.

Supernatant derived from a strain of Fructobacillus capable of promoting core microbiome development in honey bees can also be provided to a colony to act as a honey bee probiotic. Without wishing to be bound by any particular theory, it is believed that the supernatant comprises fermentative products, such as lactic acid CO₂, ethanol, and acetate, which can selectively promote the growth of the honey bee specific microbial community. This is at least in part supported by the fact that selective culture of many of the “core” microbiome members requires elevated atmospheric CO₂ and acidic media such as MRS.

In place of, or in conjunction with, one or more probiotic strains of Fructobacillus, the supernatant of one or more strains of Fructobacillus capable of promoting core microbiome development in honey bees can be provided to a colony.

Co-cultured with five isolates (Firm-5 D7-1, Firm-4 G10-1, Bifido G10-2, Firm-4 SF6D, and Bifido B08), Fructobacillus strain FJL (labeled as “F2” throughout the drawings) significantly promoted growth above the expected optical density, demonstrating this particular strain's ability to promote microbiome development in honey bees. Fructobacillus FJL can be provided to a colony to promote core microbiome development in honey bees either on its own, or in combination with one or more additional probiotic strains.

As described above, antibiotic resistant mutants can be isolated from Fructobacillus strains identified as having probiotic properties. This is true for strain FJL; tetracycline resistant FJL mutants can be identified by growing FJL on media comprising tetracycline and selecting those colonies capable of growing on the media. Tetracycline resistant FJL mutants can be provided to honey bee colonies to promote core microbiome development in honey bees.

Applicant will deposit Fructobacillus strain FJL with the American Type Culture Collection (ATCC), Manassas, Va., in compliance with the Budapest Treaty and in compliance with 37 C.F.R. §1.801-§1.809. The ATCC Accession No. will be provided upon receipt thereof. Following deposit with the ATCC, access to this deposit will be available during the pendency of this application to persons determined by the Commissioner of Patents and Trademarks to be entitled thereto under 37 CFR §1.14 and 35 USC §122.

Prior to being provided to the colony, one or more strains of Fructobacillus capable of promoting microbiome development in honey bees, the one or more strains are generally formulated into a composition. Generally, the composition will comprise at least one strain of probiotic Fructobacillus or a supernatant derived therefrom, and an acceptable carrier. Such a composition can be in the form of, for example, a liquid suspension, a paste, a syrup, or a gel. Probiotic strains of Fructobacillus, or methods to identify them, are described above. An acceptable carrier should be non-toxic to the Fructobacillus and to honey bees, and can also include an ingredient that promotes viability of the microorganism during storage. The carrier can be, for example, a liquid carrier or gel-based carrier, which are well known in the art. Such carriers include, but are not limited to, water, physiological electrolyte solutions, and glycols such as methanol, ethanol, propanol, butanol, ethylene glycol, and propylene glycol.

The composition can further comprise one or more carbon sources as a nutrient source for the honey bees, such as fructose, glucose, sucrose, maltose, galactose, sorbitol, xylan, pectin, and lignin. In particular examples, the carbon source is at least one of sucrose, fructose, and glucose.

Probiotic Fructobacillus-comprising compositions are manufactured by culturing one or more strains of Fructobacillus described above as capable of promoting microbiome development in honey bees herein, or a supernatant derived therefrom, with an appropriate carrier and a carbon source. The composition will generally be homogenous.

Compositions described herein can be provided to a honey bee colony. This can be done via feeding, wherein an effective amount of the composition is placed in or near a honey bee colony's hive so that the honey bees can feed on the composition. Methods for feeding honey bees are well known in the art, and include, for example, utilizing a frame feeder, a simple shallow tray, a bag feeder, or a jar feeder. Where the composition comprises a gel-based carrier, or is formulated as a syrup, the composition can be applied directly one or more of the frames of the colony's hive. Application to the frames of the hive allows nurse bees to have direct access to the probiotic composition.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### Examples

The materials, methods, and embodiments described herein are further defined in the following Examples. Certain embodiments of the present invention are defined in the Examples herein. It should be understood that these Examples, while indicating certain embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example I

### Methods

Bee Sampling and Microbiological Protocols.

Samples were obtained from three healthy, established hives located in Bloomington, Ind. All sampling was performed aseptically. Sterilized collection equipment and cryogenic vials were used and gloves worn throughout. Young worker bees, associated with brood cells and observed actively feeding a larva, were identified as nurse bees and collected, along with the associated larva. A sterile swab was used to sample the brood cell contents previously inhabited by the larva. Additionally, a sample of pollen, found in the nearby comb, was taken from each hive. For two hives, nectar was sampled by pipetting 100 μL of volume out of cells and into a sterile cryogenic vial. Nectar was identified for sampling as fresh honey, regurgitated by forager bees but not yet desiccated or capped for maturation. Each of these five samples (Nurse, Larvae, Cell, Pollen, and Nectar) were collected from the same frame within each individual hive. All samples were transported on ice and directly plated on media within ½ hour of collection.

Nurse bee guts were removed via aseptic hindgut dissection, and homogenized using a plastic, sterile pestle in 1× Phosphate Buffered Saline solution (PBS, pH 8.0). Bee bread and whole larval samples were similarly homogenized. Each homogenate was plated on de Man, Rogosa, and Sharpe (MRS) agar at 1:100 dilutions. Swabs taken from cells were streaked directly, without dilution. Cultures were incubated anaerobically at 37° C. for 24 hours. The resulting cultures of bacteria on each plate were scraped from the plate in 1 mL PBS, and then pipetted into 1.5 mL microcentrifuge tubes. The use of pH neutral PBS did not bias results, as it was possible to culture representative isolates of Fructobacillus, firm-4, firm-5, Bifida and Lactobacillus on neutral to slightly basic media (LB, BHI, and TSA) in addition to MRS.

DNA Extraction, Amplification, and Sequencing.

DNA was extracted from the bacterial homogenates using the MoBio PowerSoil DNA extraction kit. DNA concentration from each sample was quantified spectrophotometrically, normalized, and amplified via polymerase chain reaction using Earth Microbiome barcoded primers, 515F and 806R, tags rcbc1-20 (Caporaso et al. (2012) Isme J, 6:1621-1624). Earth Microbiome amplification protocols were followed, except for the polymerase used (NEB HF Phusion). Reactions were performed in triplicate, using 100 ng of template DNA for each 25 μl reaction. Each reaction was visualized on a 1% agarose gel to confirm amplification. Replicate amplicons were pooled, and then cleaned with a Qiagen PCR cleanup kit. Picogreen protocol was used to quantify DNA concentration for each pooled sample. Samples were then normalized and pooled collectively for sequencing. Sequencing was performed on an Illumina Miseq, using 300 PE cycles.

Bioinformatics and OTU Based Analyses.

All sequence processing was performed using the Mothur microbial ecology suite (Schloss et al. (2009) Appl Environ Microbiol, 75:7537-7541). Reads from each sample were combined into contiguous sequences, and screened for quality (maxambig=0, maxlength=275). Sequences were then aligned to the Silva reference database (silva.bacteria.fasta), preclustered, and examined for chimeras via the uchime function. Following removal of chimeric sequences, sequences were taxonomically classified using a honey bee specific training set as a reference (Newton and Roeselers, (2012) Bmc Microbiol, 12), and binned into Operational Taxonomic Units (OTUs) based upon 99% sequence identity. The data set was also subsampled to the smallest sample size of 4186 sequences, in order to normalize results across all environments. The ten OTUs with the highest sequence abundance in this subsampled data set were identified (Table 1). Data from these ten OTUs, from the three sampled hives, were averaged for each environment, and standard errors were calculated. Additionally, relative sequence abundance was explored for each hive independently. Diversity metrics (such as Simpson indices, Bray-Curtis dissimilarities) were also calculated within Mothur.

Network Analysis.

Presence of each of the top ten OTUs was calculated for each sampling environment (nodes) and used to generate an interaction network in Cytoscape. For visualization purposes only, the connections between nodes (edges) were weighted based on relative abundance of the shared OTU making up that edge. The network was constructed using OTU data from two of the three sampled hives, for which we had data for all six sampled environments. To identify important hubs in the network, centrality was assessed. Betweenness centrality measures how often a path passes through a specific node while moving from one node to another (Szalay-Beko et al. (2012) Bioinformatics, 28:2202-2204).

TABLE 1
Ten OTUs with the highest sequence abundance.

| | Mothur | | Accession | % |
|---|---|---|---|---|
| OTU | classification | Top BLAST hit | Number | Identity |
| OTU0001 | Fructobacillus | Fructobacillus | JX896573.1 | 100% |
| | | fructosus | | |
| OTU0002 | Firm-4 | Lactobacillus sp. | JX896491.1 | 100% |
| OTU0003 | Firm-5 | Lactobacillus sp. | JX896473.1 | 100% |
| OTU0025 | Firm-5 | Lactobacillus sp. | JX896461.1 | 100% |
| OTU0039 | Bacillus | Bacillus | CP010088.1 | 100% |
| | weihenstephanensi | thuringiensis | | |
| OTU0072 | Alpha 2.2 | Acetobacteraceae | KM454405.1 | 100% |
| OTU0092 | Lactobacillales | Lactobacillus | KM454401.1 | 100% |
| | | kunkeei | | |
| OTU0107 | Bifidobacteriaceae | Bifidobacterium | KM454415.1 | 100% |
| | | asteroids | | |
| OTU0115 | Firm-5 | Lactobacillus sp. | JX896483.1 | 100% |
| OTU0412 | Firm-5 | Lactobacillus sp. | JX896514.1 | 100% |

Bacterial Culture, Antibiotic Sensitivity, and Co-Culture Assays.

The Newton Laboratory honey bee bacterial strain bank was utilized as a source of bacterial isolates for this portion of the work. Bacteria from the honey bee gut and bee bread were cultured on either MRS, LB, BHI or TSA agar (37° C. for 48 hours under anaerobic conditions) and individual colonies were massively isolated using a robotic colony picker (QPExpression, Genetix). The classification of each isolate was based on 16S rRNA gene sequencing and classification using the Naïve Bayesian Classifier and the honey bee specific training set. For this study, LAB isolates identified in each of the sampled environments (Bifidobacteria, Firm-4, Firm-5, and Fructobacillus) were chosen.

Each isolate was cultured for 48 hours in MRS broth at 37° C. under anaerobic conditions. After 48 hours, culture OD600 measurements were taken and each was normalized to the lowest optical density. The bacteria were cultured alone or in co-culture in triplicate, parallel experimental replicates under all pairwise combinations. In addition, bacterial supernatants were used in lieu of cultures to determine if metabolic by-products of a specific organism (Fructobacillus) stimulated growth of isolates. To analyze the co-culture data, first the expected optical density of co-cultures was calculated based on the growth of each isolate alone. If isolates grew better in co-culture, the expected optical density would be significantly above (that is outside of the standard deviation) of the calculated expected OD. Similarly, if one of the isolates inhibited the growth of the other, the OD would be below the expected value. Optical density measured above or below the standard deviation of the expected value was considered significant.

Experiments using culture supernatants were similarly analyzed (using Microsoft Excel). To examine a specific interaction between Fructobacillus spent medium and the honey bee specific Firm-5 isolate, Firm-5 was grown to an optical density of 1.4 and sub-cultured to an OD of 0.01 in either MRS or Fructobacillus spent medium (MRS medium in which Fructobacillus had been cultured at 30° C., aerobically, to an OD600 of 1.5 then spun at 15,000 RPM for 10 minutes to remove cells). After 48 hours of growth at 37° C. under anaerobic conditions, OD600 measurements were taken and cultures were diluted and plated. All results were normalized to starting OD600 values.

Evolutionary analyses were conducted in MEGA6 using the 16S rRNA gene sequences and using a Maximum Likelihood method based on the General Time Reversible Model with a gamma distribution, invariable sites and 100 bootstrap replicates (Tamura et al. (2013) Mol Biol Evol, 30:2725-2729). For antibiotic sensitivity tests, overnight cultures of Fructobacillus sp. were mixed with soft MRS agar and overlayed onto MRS plates onto which an antibiotic impregnated disc had been placed. Diameters of zones of inhibition were measured around each antibiotic disc using a ruler. Antibiotics used were BBL Sensi-Disc tetracycline 30 mcg, ampicillin with sulbactam 20 mcg, rifampin 5 mcg, ciprofloxacin 5 mcg, and vancomycin 30 mcg.

Selection of Tetracylcine Resistant Fructobacillus.

Fructobacillus was grown on MRS agar overnight at 30° C. under aerobic conditions. A single isolated colony was then restreaked onto MRS agar containing tetracycline (50 mg/mL). Isolated colonies growing on tetracycline containing medium were identified as Fructobacillus using polymerase chain reaction (PCR) targeting the 16S rRNA gene.

Carbon Source Utilization Assay.

The same Fructobacillus isolate as used for co-culture assays was grown for 72 hours in MRS broth at 30° C. under aerobic conditions. Cells were pelleted via centrifugation and the supernatant was removed. The pellet was resuspended in a buffer of 0.1M Tris-HCl (pH 6.5). This centrifugation and resuspension process was performed twice to ensure removal of all residual MRS. 15 μl of cell suspension was added to each well of a Biolog MT2 plate (Biolog Inc.). To this, 150 μl of 2% carbohydrate solutions were added to the wells, with each carbohydrate repeated in triplicate. Additionally, water control wells were established in triplicate, inoculated with cell suspension and filtered, autoclaved water. The carbon sources examined in this study were fructose, glucose, sucrose, maltose, galactose, sorbitol, xylan, pectin, and lignin (all sourced from Sigma-Aldrich). Plates were read via spectrophotometry at A590 immediately following inoculation in order to establish as baseline. Plates were incubated aerobically at 30° C., and A590 readings were taken every 24 hours. The assay was deemed complete when a maximum A590 was observed for the plates. To assess whether a carbon source was utilized, absorbance readings from time points with peak absorbance were compared to absorbance of the initial time point. Absorbance in water control wells was subtracted from absorbance in carbohydrate containing wells. These differences were averaged. Using standard unpaired T-tests, differences in growth compared to the water control were compared between initial and final time points. A carbon source was determined to have been utilized by Fructobacillus if the difference was statistically significant with a p-value of 0.001 or lower.

### Example II

### LAB Community Profiles Across Environments

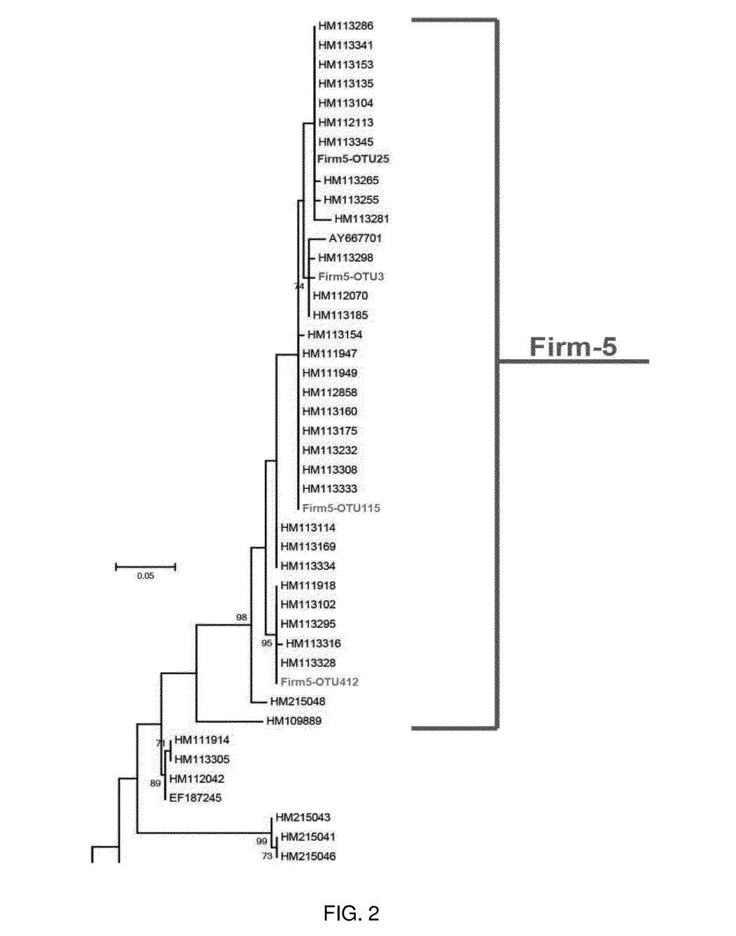

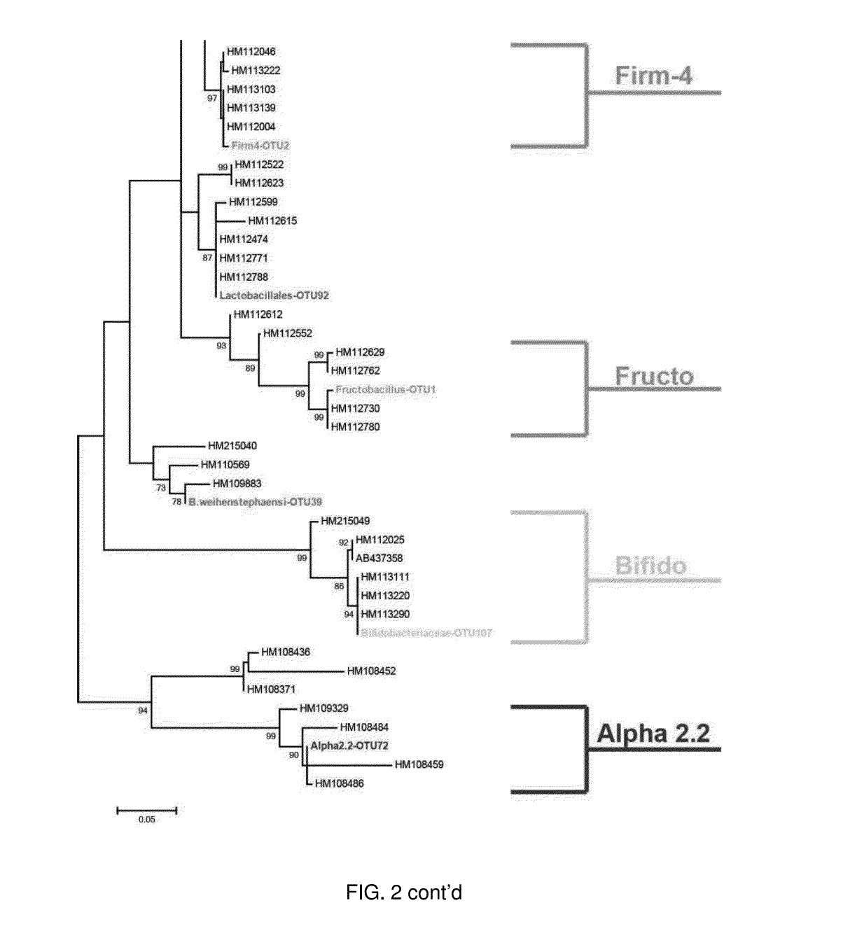

Lactic acid bacteria associated with the honey bee was chosen as a representative community, through which potential trends in microbial transmission between honey bee-associated environments could be examined Processing of 2,040,169 total sequences resulted in 1,519,195 unique sequences, grouped into 4,005 individual Operational Taxonomic Units (OTUs) when binned at 99% sequence identity. The rationale behind using a 99% identity threshold was to reach strain level resolution when examining each environment. This facilitated the ability to determine if specific microbes were being transferred between environments, or if the appearance of the same taxa in two locations was merely an artifact of homology. An abundance threshold of 1% of total sequence abundance was applied to the data set, yielding ten OTUs that met the criteria. These ten OTUs dominated the data set, containing 89.7% of total sequence abundance. The other 10.3% of OTUs in the sequence data were similarly classified as the ten largest OTUs (as Firm-5, Fructobacillus, Bacillus weihenstephaensi, Bifidobacteriaceae, Firm-4, Lactobacillales, or Alpha2.2), but did not meet the abundance threshold. To confirm that these top ten OTUs were members of a group of bacteria previously identified as associated with the honey bee gut, a phylogenetic analysis was performed. Utilizing representative sequences taken from each OTU (the most abundant sequence in that OTU), as well as sequences from a honey bee specific training set (Newton and Roeselers, (2012) Bmc Microbiol, 12), a maximum likelihood tree was constructed. The phylogeny confirmed the classification of each of the top ten OTUs, with each member forming a clade with previously identified sequences (FIG. 2). The combined culture and amplicon sequencing method successfully identified previously known honey bee-associated bacteria.

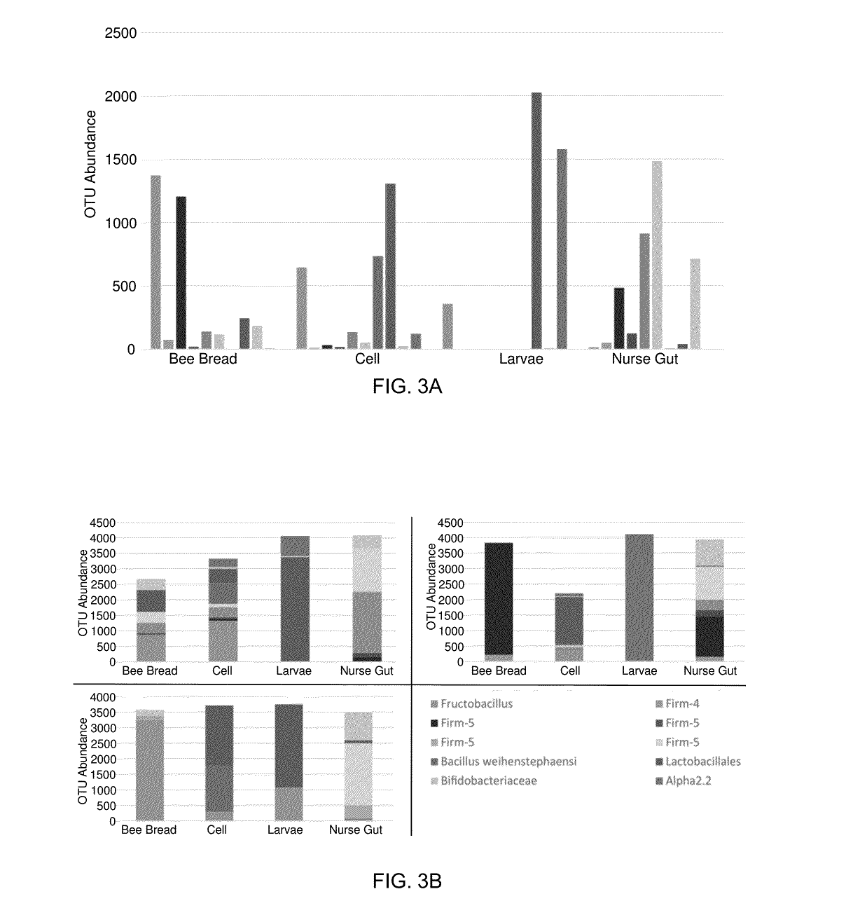

Community richness and diversity were assessed for each sampled environment, using the Chao 1 richness index and the Inverse Simpson index, respectively. Upon averaging across each of the three sampled hives, it was found that no sampled library contained a significantly richer or more diverse culturable LAB community (Table 2). To identify trends in culturable LAB composition across sampling environments, abundances of the top ten OTUs were averaged and analyzed for all hives together as well as independently (FIGS. 3A-3B). The culturable LAB community profile in larvae was different as compared to nurse bees—Lactobacillales, Alpha 2.2, and Fructobacillus largely representative of larval samples contrasted with a predominantly Firm-5 and Bifidobacteriaceae LAB community culturable from the nurse gut (FIG. 3A). The same Lactobacillales and Fructobacillus OTUs found in larvae were also identified in the bee bread and the brood cell (FIG. 3), as well as in nectar samples (number of sequences/total; Fructobacillus=2257/4186; Lactobacillales=1320/4186). While a significant amount of variation was observed in larval LAB communities from different hives, nurse guts demonstrated largely consistent compositions (Bray-Curtis dissimilarity: Mean±SD=0.192±0.0.029) (Table 2).

TABLE 2
Microbial community richness and diversity measures for each sampled
environment in the honey bee hive. Each metric was calculated for
individual replicates and averaged within each environment. Chao1
estimates show expected taxa richness within each environment. Inverse
Simpson is a measure of diversity, with a higher number indicating
greater diversity. Bray-Curtis represents the dissimilarity found
in pairwise comparisons between samples from the same environment
and highlights the consistency of the nurse gut replicates.

| | | | | | Bray- | |
|---|---|---|---|---|---|---|
| Sample | Chao1 | StDev | InvSimpson | StDev | Curtis | StDev |
| Bee Bread | 37.667 | 23.544 | 2.2372 | 1.7716 | 0.8008 | 0.2033 |
| Brood | 90.503 | 66.414 | 4.1760 | 1.6069 | 0.5985 | 0.0996 |
| Cell | | | | | | |
| Larvae | 12.083 | 13.220 | 1.7666 | 0.5928 | 0.6842 | 0.2725 |
| Nurse Gut | 10.000 | 8.8882 | 1.4677 | 0.3536 | 0.1921 | 0.0286 |

### Example III

### Environmental Habitats Facilitate Transfer of Bacteria Across the Colony

To examine OTU based relationships between sampling environments, an interaction network was generated. The presence of identical OTUs in two different habitats suggests microbial exchange between the two habitats. By using a classification threshold of 99%, the likelihood that the same strain was being observed in the two different environments was increased. If two habitats undergo frequent and extensive microbial exchange, it would expected that a large number of OTUs would be shared. Through pairwise comparisons of environments containing identical OTUs, connections (edges), weighted based on proportion of total sequence abundance observed, were made between environments (nodes). Visual inspection and connectivity analysis demonstrated the presence of homogeneity in interactions; microbes sampled were found across virtually all hive environments, showing that these environments are highly connected (FIG. 5). Environments that are behaviorally connected, such as the Nurse Gut and the Brood Cell, are also connected in this interaction network. Additionally, Betweenness centrality metrics point to both the brood cell and bee bread as central hubs of the network, through which OTUs are connected between environments (Betweenness centrality: Brood Cell=1.97; Bee Bread=1.822) (FIG. 4). These environments act as microbial hubs through which honey bees obtain, deposit, and propagate lactic acid bacteria within the hive and to the next generation.

The network analysis was also performed using a 97% classification threshold in order to reinforce results yielded from the 99% classified network. Although the network was less well resolved, and connectivity and centrality measurements were quantitatively different, trends in the data remained unchanged (Brood Cell and Bee Bread maintained the highest Betweenness centrality, while connectivity was equally distributed across environments). This showed that results were not biased based on the OTU divergence threshold used.

### Example IV

### Fructobacillus is Present Early in Bee Development, Found in Bee-Associated Environments, and Promotes the Growth of Honey Bee Specific Microbes

Larvae are in contact with both the brood cell and the bee bread during development and these hive components are known to efficiently transmit Firmicutes. As Fructobacillus and Lactobacillus sp. were found in the brood cell and the bee bread, both microbial hubs based on our analyses, it was sought to determine if honey bee core members interacted in vitro with these two taxa. Co-culture assays were use, and it was found that the “non-core”, yet predominant taxa found associated with the microbial hubs (the brood cell and the bee bread) promoted the growth of bee specific “core” members (FIG. 5). Specifically, Fructobacillus FJL (F2) in co-culture with five isolates (Firm-5 D7-1, Firm-4 G10-1, Bifido G10-2, Firm-4 SF6D, and Bifido B08) significantly promoted growth above the expected optical density. Additionally, Lactobacilliales incertae sedis G10-3 was also associated with positive growth of four isolates (Firm-5 D7-1, Firm-4 G10-1, Firm-4 SF6D, and Bifido B08). In contrast, a honey bee isolate from the Newton Lab strain bank not found associated with the hub (Staphylococcus EBHJ0) was associated with negative growth of two isolates when grown in co-culture (Firm-4 G10-1 and Bifido G10-2). To determine if Fructobacillus FJL mediated interactions with core phyla via by-products of metabolism, these same core strains were cultured with the cell-free supernatant of Fructobacillus FJL spent cultures. The results with Fructobacillus FJL supernatant recapitulated a subset of the results from Fructobacillus FJL co-cultures; Fructobacillus FJL supernatant had a similar, positive effect on growth of Bifido G10-2, Firm-4 SF6D, Firm-5 D7-1 and Bifido B08 compared to growth of these isolates alone.

Because Firm-5 species are known to associate with second instar honey bee larvae, and therefore are present early in development, the potential interaction between a Firm-5 strain (Firm-5 D7-1) and Fructobacillus FJL was further characterized. Spent medium from Fructobacillus FJL significantly increased the differential optical density reached by Firm-5 compared to growth in MRS alone based on both optical density and CFUs (FIG. 5).

### Example V

### Carbon Source Utilization of Fructobacillus

Fructobacillus and its spent media promoted the growth of other honey bee microbiome members, specifically Firm-5, showing that Fructobacillus plays a syntrophic role, interacting with other bacterial members via metabolic byproducts. The isolate's ability to utilize an array of single carbohydrate sources was thus characterized. Using Biolog MT2 plates inoculated in triplicate with the Fructobacillus FJL isolate and a panel of single simple or complex carbohydrates typically found in the honey bee's diet (see Methods), it was determined that Fructobacillus is capable of utilizing the simple sugars fructose and glucose, in addition to lignin, a plant derived complex polysaccharide (compared to water-only controls, unpaired t-test; p≦0.001 for OD590 measurements post incubation).

### Example VI

### Fructobacillus is Sensitive to Antibiotics

Honey bees are commonly prophylactically treated with oxytetracycline for the prevention of foulbrood diseases caused by the bacteria Melissococcus plutonius and Paenibacillus larvae. Data presented herein show that Fructobacillus produces byproducts that promote the growth of honey bee gut core microbiome members. The bacterial strain's resistance to antibiotics was determined, as treatment might alter the abundance of Fructobacillus in the hive. Using soft agar overlays, Fructobacillus cultures were exposed to five different antibiotics (tetracycline, ampicillin (with sulbactam), rifampicin, ciprofloxacin, and vancomycin), and the resulting zones of inhibition were measured (Table 3). In three out of five cases, Fructobacillus was found to be susceptible to these antibiotics. It was most sensitive (the largest zone of inhibition was produced) to tetracycline or ampicillin exposure. These two antibiotics are commonly used in agriculture.

TABLE 3
Antibiotic sensitivity of isolated Fructobacillus sp. The zones of
inhibition and concentrations of antibiotics used on Fructobacillus
soft agar overlays where results demonstrate marked sensitivity to
most antibiotics.

| | Concentration | Zone of Inhibition | |
|---|---|---|---|
| Antibiotic | (mcg) | (mm) | Interpretation |
| Tetracycline | 30 | 32 | Susceptible |
| Ampicillin with | 20 | 36 | Susceptible |
| Sulbactam | | | |
| Rifampin | 5 | 25 | Susceptible |
| Ciprofloxacin | 5 | 9 | Resistant |
| Vancomycin | 30 | 0 | Resistant |

While the invention has been described with reference to various embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiments disclosed herein contemplated for carrying out this invention, but that the invention will include all embodiments falling within the scope of the claims.

1. A method of promoting microbiome development in honey bees, comprising: providing an effective amount of one or more strains of Fructobacillus capable of promoting microbiome development in honey bees, or a supernatant thereof, to a honey bee colony.

2. The method of claim 1, wherein at least one of the one or more strains of Fructobacillus is resistant to tetracycline.

3. The method of claim 1, wherein the one or more strains of Fructobacillus is chosen from at least one of: strains of Fructobacillus exogenous to the honey bee colony; and strains of Fructobacillus endogenous to the honey bee colony.

4. The method of claim 1, wherein the one or more strains of Fructobacillus is provided to the honey bee colony by at least one technique chosen from feeding the one or more strains of Fructobacillus to honey bees of the honey bee colony, and applying the one or more strains of Fructobacillus to one or more frames of the honey bee colony's hive.

5. The method of claim 1, wherein the one or more strains of Fructobacillus is formulated into a composition prior to being fed or applied.

6. The method of claim 5, wherein the composition comprises a carrier chosen from a liquid carrier and gel-based carrier.

7. The method of claim 1, wherein the composition comprises at least one carbon source chosen from fructose, glucose, and lignin.

8. The method of claim 1, wherein one of the one or more strains of Fructobacillus is Fructobacillus strain FJL, a representative sample of the strain FJL having been deposited under ATCC Accession No. PTA-122552.

9. The method of claim 1, wherein one of the one or more strains of Fructobacillus is a tetracycline resistant mutant of Fructobacillus strain FJL, a representative sample of the strain FJL having been deposited under ATCC Accession No. PTA-122552.

10. A composition comprising: one or more strains of Fructobacillus capable of promoting microbiome development in honey bees, or a supernatant thereof, and a carrier.

11. The composition of claim 10, wherein the carrier is chosen from a liquid carrier and a gel-based carrier.

12. The composition of claim 10, wherein at least one of the one or more strains of Fructobacillus is resistant to tetracycline.

13. The composition of claim 10, further comprising at least one carbon source chosen from fructose, glucose, and lignin.

14. The composition of claim 10, wherein one of the one or more strains of Fructobacillus is Fructobacillus strain FJL, a representative sample of the strain FJL having been deposited under ATCC Accession No. PTA-122552.

15. The composition of claim 10, wherein one of the one or more strains of Fructobacillus is a tetracycline resistant mutant of Fructobacillus strain FJL, a representative sample of the strain FJL having been deposited under ATCC Accession No. PTA-122552

16. A process for manufacturing the composition of claim 10, comprising: culturing one or more strains of Fructobacillus capable of promoting microbiome development in honey bees, and combining at least one of the obtained culture(s) or a supernatant thereof, with a carrier chosen from a liquid carrier and a gel-based carrier, and at least one carbon source chosen from fructose, glucose, and lignin into a homogenous composition.

17. The process of claim 16 wherein at least one of the one or more strains of Fructobacillus is resistant to tetracycline.

18. The process of claim 16, wherein the one or more strains of Fructobacillus is chosen from at least one of: strains of Fructobacillus exogenous to a honey bee colony; and strains of Fructobacillus endogenous to a honey bee colony.

19. The process of claim 16, wherein one of the one or more strains of Fructobacillus is Fructobacillus strain FJL, a representative sample of the strain FJL having been deposited under ATCC Accession No. PTA-122552.

20. The process of claim 16, wherein one of the one or more strains of Fructobacillus is a tetracycline resistant mutant of Fructobacillus strain FJL, a representative sample of the strain FJL having been deposited under ATCC Accession No. PTA-122552.

21. (canceled)
